(19) United States
(12) Patent Application Publication
(10) Pub. No.: US 2005/0106602 A1
(43) Pub. Date: May 19, 2005

(54) Simplified methods for isolating nucleic acids from cellular materials

(76) Inventor: Hashem Akhavan-Tafti, Howell, MI (US)

Correspondence Address: LUMIGEN, INC., 22900 W. EIGHT MILE ROAD, SOUTHFIELD, MI, 48034, US

(21) Appl. No.: 10/942,491

(22) Filed: Sep. 16, 2004

Related U.S. Application Data
(63) Continuation-in-part of application No. 10/714,763, filed on Nov. 17, 2003.
(63) Continuation-in-part of application No. 10/715,284, filed on Nov. 17, 2003.

(51) Int. Cl. C12Q 1/68; C07H 21/02; C07H 21/04

(52) U.S. Cl. 435/6; 536/25.4

(57) ABSTRACT

Methods of isolating nucleic acids from samples of cellular material are disclosed which use solid phase binding materials and which avoid the use of a lysis solution. The use of the solid phase binding materials unexpectedly allow the nucleic acid content of cells to be freed and captured directly and in one step. The new methods represent a significant simplification over existing methods. Preferred solid phase materials for use with the methods and compositions of the invention comprise a quaternary onium nucleic acid binding portion.

### CROSS REFERENCE TO RELATED APPLICATION

The present application is a continuation-in-part of Applicants' co-pending U.S. application Ser. No. 10/714,763, filed on Nov. 17, 2003 and U.S. application Ser. No. 10/715,284, filed on Nov. 17, 2003.

### FIELD OF THE INVENTION

The present invention relates to simplified methods for capturing and isolating nucleic acids, particularly total genomic nucleic acid from materials of biological origin.

### BACKGROUND OF THE INVENTION

Molecular diagnostics and modern techniques in molecular biology (including reverse transcription, cloning, restriction analysis, amplification, and sequence analysis), require the extraction of nucleic acids. Obtaining nucleic acid is complicated by the complex sample matrix in which target nucleic acids are found. Such samples include, e.g., cells from tissues, cells from bodily fluids, blood, bacterial cells in culture, agarose gels, polyacrylamide gels, or solutions resulting from amplification of target nucleic acids. Sample matrices often contain significant amounts of contaminants which must be removed from the nucleic acid(s) of interest before the nucleic acids can be used in molecular biological or diagnostic techniques.

Conventional techniques for obtaining target nucleic acids from mixtures produced from cells and tissues as described above, require the use of hazardous chemicals such as phenol, chloroform, and ethidium bromide. Phenol/chloroform extraction is used in such procedures to extract contaminants from mixtures of target nucleic acids and various contaminants. Alternatively, cesium chloride-ethidium bromide gradients are used according to methods well known in the art. See, e.g., Molecular Cloning, ed. by Sambrook et al. (1989), Cold Spring Harbor Press, pp. 1.42-1.50. The latter methods are generally followed by precipitation of the nucleic acid material remaining in the extracted aqueous phase by adding ethanol or 2-propanol to the aqueous phase to precipitate nucleic acid. The precipitate is typically removed from the solution by centrifugation, and the resulting pellet of precipitate is allowed to dry before being resuspended in water or a buffer solution for further use.

Simpler and faster methods have been developed which use various types of solid phases to separate nucleic acids from cell lysates or other mixtures of nucleic acids and contaminants. Such solid phases include chromatographic resins, polymers and silica or glass-based materials in various shapes and forms such as fibers, filters and coated containers. When in the form of small particulates, magnetic cores are sometimes provided to assist in effecting separation.

Kits containing a solid binding support material have been developed and are available commercially for use in methods of isolating genomic from bacterial culture and from whole human blood. Procedures provided by the manufacturers invariably specify that cells must be lysed before commencing with removal and purification of the nucleic acid. An additional precipitation step is sometimes also employed before use of the solid support (e.g., K. Smith, et al., J. Clin. Microbiol., 41(6), 2440-3 (2003); P. Levison, et al., J. Chromatography A, 827, 337-44 (1998)).

One type of solid phase used in isolating nucleic acids comprises porous silica gel particles designed for use in high performance liquid chromatography (HPLC). The surface of the porous silica gel particles is functionalized with anion-exchangers to exchange with plasmid DNA under certain salt and pH conditions. See, e.g. U.S. Pat. Nos. 4,699,717, and 5,057,426. Plasmid DNA bound to these solid phase materials is eluted in an aqueous solution containing a high concentration of a salt. The nucleic acid solution eluted therefrom must be treated further to remove the salt before it can be used in downstream processes.

Other silica-based solid phase materials comprise controlled pore glass (CPG), filters embedded with silica particles, silica gel particles, diatomaceous earth, glass fibers or mixtures of the above. Each silica-based solid phase material reversibly binds nucleic acids in a sample containing nucleic acids in the presence of chaotropic agents such as sodium iodide (NaI), guanidinium thiocyanate or guanidinium chloride. Such solid phases bind and retain the nucleic acid material while the solid phase is subjected to centrifugation or vacuum filtration to separate the matrix and nucleic acid material bound thereto from the remaining sample components. The nucleic acid material is then freed from the solid phase by eluting with water or a low salt elution buffer. Commercially available silica-based solid phase materials for nucleic acid isolation include, e.g., Wizard™ DNA purification systems products (Promega, Madison, Wis.), the QiaPrep™ DNA isolation systems (Qiagen, Santa Clarita, Calif.), High Pure (Roche), and GFX Micro Plasmid Kit, (Amersham).

Polymeric resins in the form of particles are also in widespread use for isolation and purification of nucleic acids. Carboxylate-modified polymeric particles (Bangs, Agencourt) are known. Polymers having quaternary ammonium head groups are disclosed in European Patent Application Publ. No. EP 1243649A1. The polymers are inert carrier particles having covalently attached linear non-crosslinked polymers. This type of polymeric solid phase is commonly referred to as a tentacle resin. The linear polymers incorporate quaternary tetraalkylammonium groups. The alkyl groups are specified as methyl or ethyl groups (Column 4, lines 52-55). Longer alkyl groups are deemed undesirable.

Other solid phase materials for binding nucleic acids based on the anion exchange principle are in present use. These include a silica based material having DEAE head groups (Qiagen) and a silica-NucleoBond AX (Becton Dickinson, Roche-Genopure) based on the chromatographic support described in EP0496822B1. Polymer resins with polymeric-trialkylammonium groups are disclosed in EP 1243649 (GeneScan). Carboxyl-modified polymers for DNA isolation are available from numerous suppliers. Nucleic acids are attracted under high salt conditions and released under low ionic strength conditions. A polymeric microcarrier bead having a cationic trimethylamine exterior is described in U.S. Pat. No. 6,214,618. The beads have a relatively large diameter and are useful as a support for cell attachment and growth in culture.

Polymeric beads having a tributylphosphonium head group have been described for use as phase transfer catalysts in a three phase system. The beads were prepared from a cross-linked polystyrene. (J. Chem. Soc. Perkin Trans. II, 1827-1830, (1983)). Polymer beads having a pendant trialkylphosphonium group linked to a cross-linked polystyrene resin through alkylene chains and alkylene ether chains have also been described (Tomoi, et al., Makromolekulare Chemie, 187(2), 357-65 (1986); Tomoi, et al., Reactive Polymers, Ion Exchangers, Sorbents, 3(4), 341-9 (1985)). Mixed quaternary ammonium/phosphonium insoluble polymers based on cross-linked polystyrene resins are disclosed as catalysts and biocides (Davidescu, et al., Chem. Bull. Techn. Univ. Timisoara, 40(54), 63-72 (1995); Parvulescu, et al,. Reactive & Functional Polymers, 33(2,3), 329-36 (1997).

Magnetically responsive particles have also been developed for use as solid phases in isolating nucleic acids. Several different types of magnetically responsive particles designed for isolation of nucleic acids are known in the art and commercially available from several sources. Magnetic particles which reversibly bind nucleic acid materials directly include MagneSil™ particles (Promega). Magnetic particles are also known that reversibly bind mRNA via covalently attached avidin or streptavidin having an attached oligo dT tail for hybridization with the poly A tail of mRNA.

Various types of magnetically responsive silica-based particles are known for use as solid phases in nucleic acid binding isolation methods. One such particle type is a magnetically responsive glass bead, preferably of a controlled pore size available as Magnetic Porous Glass (MPG) particles from CPG, Inc. (Lincoln Park, N.J.); or porous magnetic glass particles described in U.S. Pat. Nos. 4,395,271; 4,233,169; or 4,297,337. Another type of magnetic particle useful for binding and isolation of nucleic acids is produced by incorporating magnetic materials into the matrix of polymeric silicon dioxide compounds. (German Patent DE4307262A1) Magnetic particles comprising iron oxide nanoparticles embedded in a cellulose matrix having quaternary ammonium group is produced commercially by Cortex Biochem (San Leandro, Calif.) as MagaCell-Q™.

Particles or beads having inducible magnetic properties comprise small particles of transition metals such as iron, nickel, copper, cobalt and manganese to form metal oxides which can be caused to have transitory magnetic properties in the presence of magnet. These particles are termed paramagnetic or superparamagnetic. To form paramagnetic or superparamagnetic beads, metal oxides have been coated with polymers which are relatively stable in water. U.S. Pat. No. 4,554,088 discloses paramagnetic particles comprising a metal oxide core surrounded by a coat of polymeric silane. U.S. Pat. No. 5,356,713 discloses a magnetizable microsphere comprised of a core of magnetizable particles surrounded by a shell of a hydrophobic vinylaromatic monomer. U.S. Pat. No. 5,395,688 discloses a polymer core which has been coated with a mixed paramagnetic metal oxide-polymer layer. Another method utilizes a polymer core to adsorb metal oxide such as for example in U.S. Pat. No. 4,774,265. Magnetic particles comprising a polymeric core particle coated with a paramagnetic metal oxide particle layer is disclosed in U.S. Pat. No. 5,091,206. The particle is then further coated with additional polymeric layers to shield the metal oxide layer and to provide a reactive coating. U.S. Pat. No. 5,866,099 discloses the preparation of magnetic particles by co-precipitation of mixtures of two metal salts in the presence of a protein to coordinate the metal salt and entrap the mixed metal oxide particle. Numerous exemplary pairs of metal salts are described. U.S. Pat. No. 5,411,730 describes a similar process where the precipitated mixed metal oxide particle is entrapped in dextran, an oligosaccharide.

Alumina (aluminum oxide) particles for irreversible capture of DNA and RNA are disclosed in U.S. Pat. No. 6,291,166. Bound nucleic acid is available for use in solid phase amplification methods such as PCR.

DNA bound to these solid phase materials is eluted in an aqueous solution containing a high concentration of a salt. The nucleic acid solution eluted therefrom must be treated further to remove the salt before it can be used in downstream processes. Nucleic acids bound to silica-based material, in contrast, are freed from the solid phase by eluting with water or a low salt elution buffer. U.S. Pat. No. 5,792,651 describes a composition for chromatographic isolation of nucleic acids which enhances the ability of the nucleic acid in transfection in cells. The composition comprises an aqueous solution containing 2-propanol and optional salts and buffer materials.

Yet other magnetic solid phase materials comprising agarose or cellulose particles containing magnetic microparticle cores are reported to bind and retain nucleic acids upon treatment with compositions containing high concentrations of salts and polyalkylene glycol (e.g. U.S. Pat. No. 5,898,071 and PCT Publication W002066993). Nucleic acid is subsequently released by treatment with water or low ionic strength buffer.

Applicants' co-pending U.S. applications Ser. Nos. 10/714,763, 10/715,284 and 10/891,880, incorporated herein by reference, disclose novel solid phase nucleic acid binding materials, including cleavable materials, and methods of binding and releasing nucleic acids.

### SUMMARY OF THE INVENTION

It is a first object of the present invention to provide methods for capturing nucleic acids from biological materials using solid phase nucleic acid binding materials without a chemical lysis step.

It is another object of the present invention to provide methods for isolating nucleic acids from biological materials by capturing the nucleic acids using solid phase nucleic acid binding materials without a chemical lysis step and subsequently releasing the captured nucleic acid.

It is an object of the present invention to provide methods for capturing and isolating nucleic acids from biological and cellular materials using solid phase nucleic acid binding materials having a cleavable linker group.

It is an object of the present invention to provide methods for capturing and isolating nucleic acids from biological and cellular materials using solid phase nucleic acid binding materials having a cationic group selected from phosphonium, ammonium and sulfonium groups.

### BRIEF DESCRIPTION OF THE DRAWINGS

FIG. 1 schematically depicts the isolation of nucleic acid from a blood sample according to the present invention.

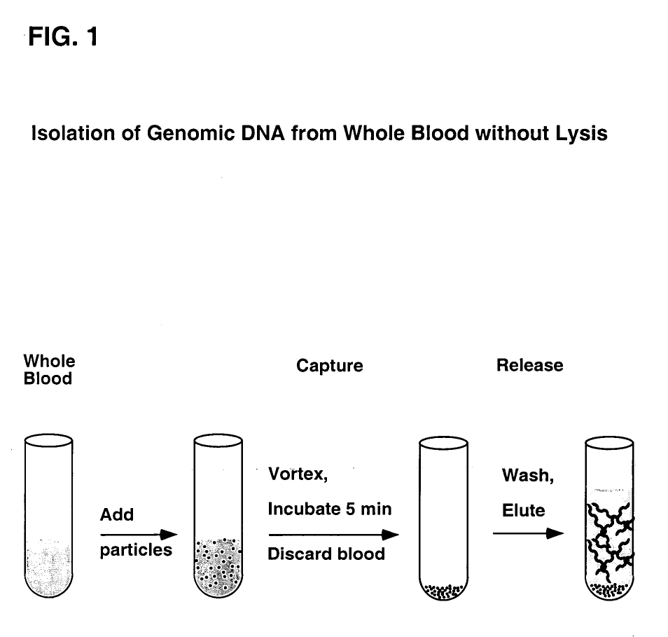

FIG. 2A is an image of a gel showing DNA isolated from human blood samples using the particles of example 1. FIG. 2B is an image of a gel showing amplification of a region of genomic DNA isolated as in FIG. 2A.

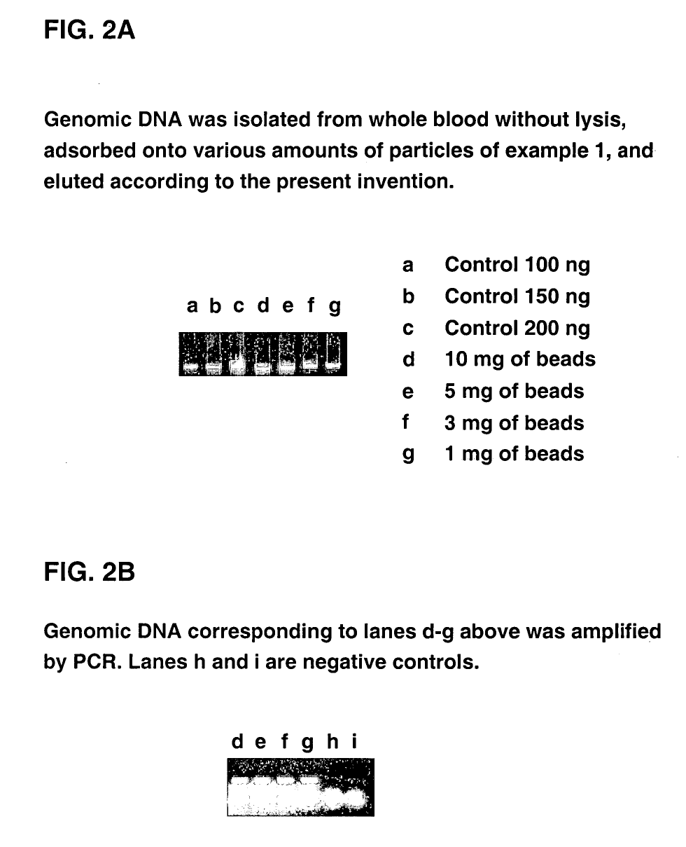

FIG. 3 is an image of a gel showing amplification of a region of genomic DNA isolated according to the present methods using the particles of example 1 or example 4 and various additives.

FIG. 4 is an image of a gel showing amplification of a region of genomic DNA isolated according to the present methods using the particles of examples 5-7.

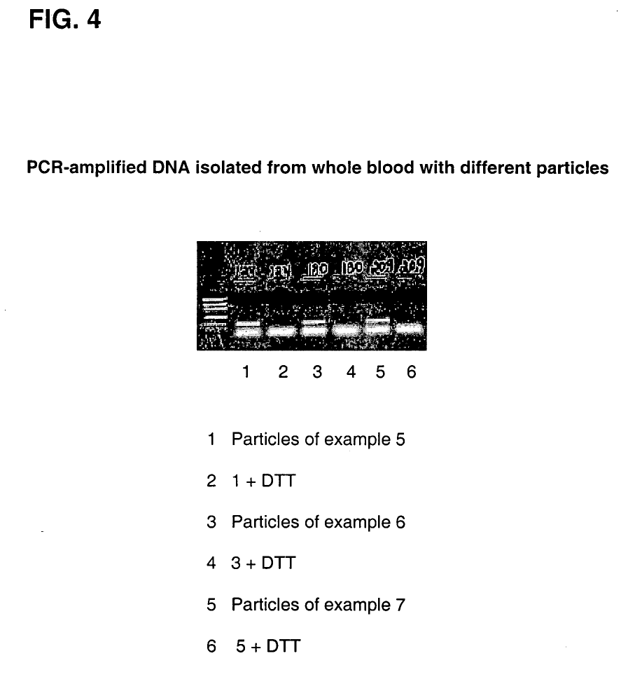

FIG. 5A is an image of a gel showing showing DNA isolated from human blood samples using the particles of examples 1 or 2, eluting with various concentrations of NaOH. FIG. 5B is an image of a gel showing amplification of a region of genomic DNA isolated as shown in 5A.

### DETAILED DESCRIPTION OF THE INVENTION

Definitions

Alkyl—A branched, straight chain or cyclic hydrocarbon group containing from 1-20 carbons which can be substituted with 1 or more substituents other than H. Lower alkyl as used herein refers to those alkyl groups containing up to 8 carbons.

Aralkyl—An alkyl group substituted with an aryl group.

Aryl—An aromatic ring-containing group containing 1 to 5 carbocyclic aromatic rings, which can be substituted with 1 or more substituents other than H.

Cellular material—intact cells or material, including tissue, containing intact cells of animal, plant or bacterial origin.

Cellular nucleic acid content—refers to nucleic acid found within cellular material and can be genomic DNA and RNA, and other nucleic acids such as that from infectious agents, including viruses and plasmids.

Magnetic particle—a particle, microparticle or bead that is responsive to an external magnetic field. The particle may itself be magnetic, paramagnetic or superparamagnetic. It may be attracted to an external magnet or applied magnetic field as when using ferromagnetic materials. Particles can have a solid core portion that is magnetically responsive and is surrounded by one or more non-magnetically responsive layers. Alternately the magnetically responsive portion can be a layer around or can be particles disposed within a non-magnetically responsive core.

Oligomer, oligonucleotide—as used herein will refer to a compound containing a phosphodiester internucleotide linkage and a 5′-terminal monophosphate group. The nucleotides can be the normally occurring ribonucleotides A, C, G, and U or deoxyribonucleotides, dA, dC, dG and dT.

Nucleic acid—A polynucleotide can be DNA, RNA or a synthetic DNA analog such as a PNA. Single stranded compounds and double-stranded hybrids of any of these three types of chains are also within the scope of the term

Release, elute—to remove a substantial portion of a material bound to the surface or pores of a solid phase material by contact with a solution or composition.

Sample—A fluid containing or suspected of containing nucleic acids. Typical samples which can be used in the methods of the invention include bodily fluids such as blood, plasma, serum, urine, semen, saliva, cell cultures, tissue extracts and the like. Other types of samples include solvents, seawater, industrial water samples, food samples and environmental samples such as soil or water, plant materials, cells originated from prokaryotes, eukaryotes, bacteria, plasmids and viruses.

Solid phase material—a material having a surface to which can attract nucleic acid molecules. Materials can be in the form of microparticles, fibers, beads, membranes, and other supports such as test tubes and microwells.

Substituted—Refers to the replacement of at least one hydrogen atom on a group by a non-hydrogen group. It should be noted that in references to substituted groups it is intended that multiple points of substitution can be present unless clearly indicated otherwise.

Nucleic acids are extracted, isolated and otherwise purified from various sample types by a variety of techniques. Many of these techniques rely on selective adsorption onto a surface of a material with some affinity for nucleic acids. After washing steps to remove other, less strongly bound components, the solid phase is treated with a solution to remove or elute bound nucleic acid(s). It is frequently necessary to extract and isolate the genomic nucleic acid from a portion of cellular material. Nucleic acids so obtained are used in subsequent processes including amplification, diagnostic tests, analysis of mutations, gene expression profiling and cloning. Samples from which nucleic acids can be isolated by the methods of the present invention include bacterial cultures, bodily fluids, whole blood and blood components, tissue extracts, plant materials, and environmental samples containing cellular materials.

Removal of cellular nucleic acid content requires the disruption or penetration of cellular membranes or walls in order to access the interior. For this purpose, prior methods employed a cell lysis step using a reagent for effecting lysis. Lysis solutions are of two types depending on the method of lysis used. One type is an aqueous solution of high pH for alkaline lysis. Another type employs one or more surfactants or detergents to disrupt cell membranes. Lysis solutions can also contain digestive enzymes such as proteinase enzymes to assist in freeing the nucleic acid content of cells. Applicants have developed methods for isolating nucleic acids from samples of cellular material using solid phase binding materials which avoid the use of a lysis solution. The solid phase binding materials unexpectedly allow the nucleic acid content of cells to be freed and captured directly and in one step. The new methods represent a significant improvement in simplicity, convenience and ease of automation since the use of lysis solutions is eliminated.

In one aspect of the invention there is provided a method of isolating nucleic acids from a sample of cellular material comprising:
a) providing a solid phase comprising: a matrix to which is attached and a nucleic acid binding portion;
b) combining the solid phase with a sample of cellular material containing nucleic acids in the absence of any added lysis solution for a time sufficient to bind the nucleic acids to the solid phase;
c) separating the sample from the solid phase; and
d) releasing the bound nucleic acids from the solid phase.

Solid phase materials for binding nucleic acids for use with the methods of the present invention comprise a matrix which defines its size, shape, porosity, and mechanical properties, and covalently linked nucleic acid binding groups and can be in the form of particles, microparticles, fibers, beads, membranes, and other supports such as test tubes and microwells. The materials further comprise a nucleic acid binding portion at or near the surface which permits capture and binding of nucleic acid molecules of varying lengths. By surface is meant not only the external periphery of the solid phase material but also the surface of any accessible porous regions within the solid phase material.

The matrix material can be any suitable substance. Preferred matrix materials are selected from silica, glass, insolublesynthetic polymers, and insoluble polysaccharides. Exemplary materials include silica based materials coated or functionalized with covalently attached surface functional groups that serve to disrupt cells and attract nucleic acids. Also included are suitably surface-functionalized carbohydrate based materials, and polymeric materials having this surface functionality. Numerous specific materials and their preparation are described in Applicants' co-pending U.S. applications Ser. Nos. 10/714,763, 10/715,284 and 10/891,880. The surface functional groups serving as nucleic acid binding groups include any groups capable of disrupting cells' structural integrity, and causing attraction of nucleic acid to the solid support. Such groups include, without limitation, quaternary ammonium and phosphonium salts and ternary sulfonium salt type materials described below.

The solid phase can further comprise a magnetically responsive portion which will usually be in the form of magnetic microparticles—particles that can be attracted and manipulated by a magnetic field. Such magnetic microparticles comprise a magnetic metal oxide or metal sulfide core, which is generally surrounded by an adsorptively or covalently bound layer to which nucleic acid binding groups are covalently bound, thereby coating the surface. The magnetic metal oxide core is preferably iron oxide or iron sulfide, wherein iron is Fe²⁺ or Fe³⁺ or both. Magnetic particles can also be formed as described in U.S. Pat. No. 4,654,267 by precipitating metal particles in the presence of a porous polymer to entrap the magnetically responsive metal particles. Magnetic metal oxides preparable thereby include Fe₃O₄, MnFe₂O₄, NiFe₂O₄, and CoFe₂O₄. Other magnetic particles can also be formed as described in U.S. Pat. No. 5,411,730 by precipitating metal oxide particles in the presence of a the oligosaccharide dextran to entrap the magnetically responsive metal particles. Yet another kind of magnetic particle is disclosed in the aforementioned U.S. Pat. No. 5,091,206. The particle comprises a polymeric core particle coated with a paramagnetic metal oxide particle layer and additional polymeric layers to shield the metal oxide layer and to provide a reactive coating. Preparation of magnetite containing chloromethylated Merrifield resin is described in a publication (Tetrahedron Lett.,40 (1999), 8137-8140).

Commercially available magnetic silica or magnetic polymeric particles can be used as the starting materials in preparing magnetic solid phase binding materials useful in the present invention. Suitable types of polymeric particles having surface carboxyl groups are known by the tradenames SeraMag™ (Seradyn) and BioMag™ (Polysciences and Bangs Laboratories). A suitable type of silica magnetic particles is known by the tradename MagneSil™ (Promega). Silica magnetic particles having carboxy or amino groups at the surface are available from Chemicell GmbH (Berlin).

When the solid phase binding material comprises an insoluble synthetic polymer portion, useful polymers are homopolymers or copolymers of one or more ethylenically unsaturated monomer units and can be crosslinked or non-crosslinked. Preferred polymers are polyolefins including polystyrene and the polyacrylic-type polymers. The latter comprise polymers of various substituted acrylic acids, amides and esters, wherein the acrylic monomer may or may not have alkyl substituents on the 2- or 3-carbon.

The nucleic acid binding (NAB) groups contained in the solid phase binding materials useful in the methods of the present invention appear to serve dual purposes. NAB groups attract and bind nucleic acids, polynucleotides and oligonucleotides of various lengths and base compositions or sequences. They also serve in some capacity to free nucleic acid from the cellular envelope. Nucleic acid binding groups include, for example, carboxyl, amine and ternary or quaternary onium groups. Amine groups can be NH₂, alkylamine, and dialkylamine groups. Ternary or quaternary onium groups include quaternary trialkylammonium groups (-QR₃⁺), phosphonium groups (-QR₃⁺) including trialkylphosphonium or triarylphosphonium or mixed alkyl aryl phosphonium groups, and ternary sulfonium groups (-QR₂⁺). The solid phase can contain more than one kind of nucleic acid binding group as described herein. Solid phase materials containing ternary or quaternary onium groups-QR₂⁺ or -QR₃⁺ wherein the R groups are alkyl of at least four carbons are especially effective in binding nucleic acids, but alkyl groups of as little as one carbon are also useful as are aryl groups. Such solid phase materials retain the bound nucleic acid with great tenacity and resist removal or elution of the nucleic acid under most conditions used for elution known in the prior art. Most known elution conditions of both low and high ionic strength are ineffective in removing bound nucleic acids. Unlike conventional anion-exchange resins containing DEAE and PEI groups, the ternary or quaternary onium solid phase materials remain positively charged regardless of the pH of the reaction medium.

In additional embodiments of the invention there is provided a method of isolating a nucleic acid from a sample comprising:
a) providing a solid phase comprising: a matrix selected from silica, glass, insoluble synthetic polymers, and insoluble polysaccharides, and an onium group attached on a surface of the matrix selected from a ternary sulfonium group of the formula QR₂⁺ X⁻ where R is selected from C₁-C₂₀ alkyl, aralkyl and aryl groups, a quaternary ammonium group of the formula NR₃⁺ X⁻ wherein the quaternary onium group wherein R is selected from C₁-C₂₀ alkyl, aralkyl and aryl groups, and a quaternary phosphonium group PR₃⁺ X⁻ wherein R is selected from C₁-C₂₀ alkyl, aralkyl and aryl groups, and wherein X is an anion,
b) combining the solid phase with a sample of cellular material containing nucleic acids in the absence of any added lysis solution for a time sufficient to bind the nucleic acids to the solid phase;
c) separating the sample from the solid phase; and
d) releasing the bound nucleic acids from the solid phase.

The step of combining the solid phase with the sample of cellular material containing nucleic acid involve admixing the sample material and the solid phase binding material and, optionally, mechanically agitating the mixture to uniformly distribute the solid phase within the volume of the sample for a time period effective to disrupt the cellular material and bind nucleic acids to the solid phase. It is not necessary that all of the nucleic acid content of the sample become bound to the solid phase, however it is advantageous to bind as much as possible. Agitation of the sample/solid phase mixture can take any convenient form including shaking, use of mechanical oscillators or rockers, vortexing, ultrasonic agitation and the like. The time required to bind nucleic acid in this step is typically on the order of a few minutes, but can be verified experimentally by routine experimentation.

The step of separating the sample from the solid phase can be accomplished by filtration, gravitational settling, decantation, magnetic separation, centrifugation, vacuum aspiration, overpressure of air or other gas to force a liquid through a porous membrane or filter mat, for example. Components of the sample other than nucleic acids are removed in this step. To the extent that the removal of other components is not complete, one or more washes can be performed to assist in their complete removal. Wash reagents to remove sample components such as salts, cellular debris, proteins, and hemoglobin include water and aqueous buffer solutions and can contain surfactants.

The step of releasing the bound nucleic acid from the solid phase involves contacting the solid phase material with a solution to release the bound nucleic acids from the solid phase. The solution should dissolve and sufficiently preserve the released nucleic acid. The solution can be a reagent composition comprising an aqueous buffer solution having a pH of about 7-9, optionally containing 0.1-3 M, buffer salt, metal halide or acetate salt and optionally containing an organic co-solvent at 0.1-50% or a surfactant.

The reagent for releasing the nucleic acid from the solid phase after cleavage can alternately be a strongly alkaline aqueous solution. Solutions of alkali metal hydroxides or ammonium hydroxide at a concentration of at least 10⁻⁴ M are effective in eluting nucleic acid from the cleaved solid phase. It is recognized that such strongly alkaline solutions are detrimental to the stability of RNA. When it is desired to obtain RNA, contact with strongly alkaline solution should be avoided or kept to a minimum time. Strongly alkaline solutions are useful in conjunction with solid phase binding materials in which the nucleic acid binding portion is attached to the matrix through a group which can be fragmented or cleaved by covalent bond breakage. Such materials are described below and in the aforementioned co-pending U.S. patent applications Ser. Nos. 10/714,763, 10/715,284 and 10/891,880.

Certain preferred embodiments employ solid phase binding materials in which the NAB groups are attached to the matrix through a linkage which can be selectively broken. Breaking the link effectively “disconnects” any bound nucleic acids from the solid phase. The link can be cleaved by any chemical, enzymatic, photochemical or other means that specifically breaks bond(s) in the cleavable linker but does not also destroy the nucleic acids of interest. Such cleavable solid phase materials comprise a solid support portion comprising a matrix selected from silica, glass, insoluble synthetic polymers, and insoluble polysaccharides to which is attached on a surface a nucleic acid binding (NAB) portion for attracting and binding nucleic acids, the NAB portion being linked by a cleavable linker portion to the solid support portion.

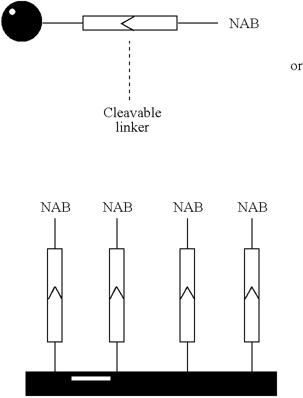

In one embodiment the NAB is a ternary onium group of the formula QR₂⁺ X⁻ or a quaternary onium group QR₃⁺ X⁻ as described above.

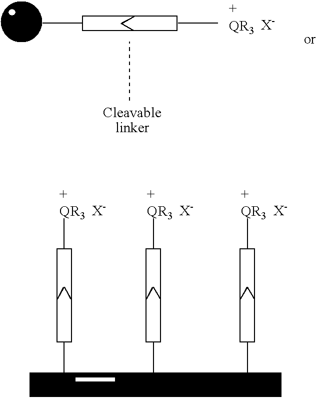

One type of cleavable solid phase is derived from commercially available polystyrene type polymers such as those of the kind referred to as Merrifield resin (crosslinked). In these polymers a percentage of the styrene units contain a reactive group, typically a chloromethyl or hydroxymethyl group as a means of covalent attachment. Replacement of some of the chlorines by reaction with a sulfide (R₂S) or a tertiary amine or phosphine produces the solid phase materials of the invention. A polymer prepared in accordance with this definition can be depicted by the formula (1) below when all of the reactive chloromethyl groups have been converted to ternary or quaternary onium groups. It is not necessary for all such groups to be converted so that polymeric solid phases of the invention will often contain a mixture of the onium group and the chloromethyl group. 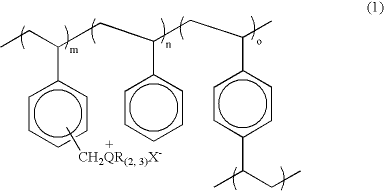
In the formula above, m, n, and o denote the mole percentage of each monomeric unit in the polymer and can take the values m from 0.1% to 100%, n from 0 to 99%, and o from 0 to 10%. More preferably m is from 1% to 20 %, n is from 80 to 99%, and o is from 0 to 10%.

In another embodiment, a cleavable solid phase is derived from a commercially available crosslinked Merrifield resin having a percentage of the styrene units contain a reactive chloroacetyl or chloropropionyl group for covalent attachment. Numerous other art-known polymeric resins can be used as the solid matrix in preparing cleavable solid phase materials. Polymeric resins are available from commercial suppliers such as Advanced ChemTech (Louisville, Ky.) and NovaBiochem. The resins are generally based on a crosslinked polymeric particle having a reactive functional group. Many suitable polymeric resins used in solid supported peptide synthesis as described in the Advanced ChemTech 2002 Catalog, pp. 105-140 are appropriate starting materials. Polymers having reactive NH₂, NH—NH₂, OH, SH, CHO, COOH, CO₂CH═CH₂, NCO, Cl, Br, SO₂CH═CH₂, SO₂Cl, SO₂NH₂, acylimidazole, oxime (C═N—OH), succinimide ester groups are each commercially available for use in preparation of polymeric solid phases of the invention. As is shown below in numerous examples and in the aforementioned co-pending patent applications, it is sometimes necessary or desirable to provide a means of covalently joining a precursor polymer resin to the ternary or quaternary onium group. This will generally comprise a chain or ring group of 1-20 atoms selected from alkylene, arylene or aralkylene groups. The chain or ring can also contain O, S, or N atoms and carbonyl groups in the form of ketones, esters, thioesters, amides, urethanes, carbonates, xanthates, ureas, imines, oximes, sulfoxides and thioketones.

The cleavable linker portion is preferably an organic group selected from straight chains, branched chains and rings and comprises from 1 to 100 atoms and more preferably from 1 to about 50 atoms. The atoms are preferably selected from C, H, B, N, O, S, Si, P, halogens and alkali metals. An exemplary linker group is a hydrolytically cleavable group which is cleaved by hydrolysis. Carboxylic esters and anhydrides, thioesters, carbonate esters, thiocarbonate esters, urethanes, imides, sulfonamides, and sulfonimides are representative as are sulfonate esters. Another exemplary class of linker groups are those groups which undergo reductive cleavage such as a disulfide (S—S) bond which is cleaved by thiols such as ethanethiol, mercaptoethanol, and DTT. Another representative group is an organic group containing a peroxide (O—O) bond. Peroxide bonds can be cleaved by thiols, amines and phosphines. Another representative group is a photochemically cleavable linker group such as nitro-substituted aromatic ethers and esters of the formula 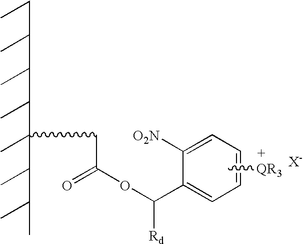
where R_{d} is H, alkyl or phenyl. Ortho-nitrobenzyl esters are cleaved by ultraviolet light according to the well known reaction below.

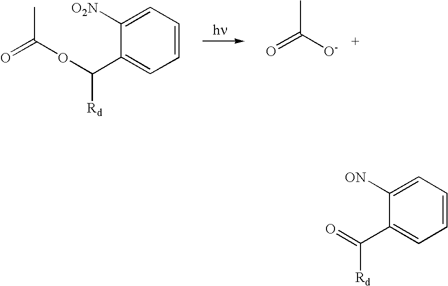

Another representative cleavable group is an enzymatically cleavable linker group. Exemplary groups include esters which are cleaved by esterases and hydrolases, amides and peptides which are cleaved by proteases and peptidases, glycoside groups which are cleaved by glycosidases.

Another representative cleavable group is a cleavable 1,2-dioxetane moiety. Such materials contain a dioxetane moiety which can be decomposed thermally or triggered to fragment by a chemical or enzymatic agent. Removal of a protecting group to generate an oxyanion promotes decomposition of the dioxetane ring. Fragmentation occurs by cleavage of the peroxidic O—O bond as well as the C—C bond according to a well known process. Cleavable dioxetanes are described in numerous patents and publications. Representative examples include U.S. Pat. Nos. 4,952,707, 5,707,559, 5,578,253, 6,036,892, 6,228,653 and 6,461,876. 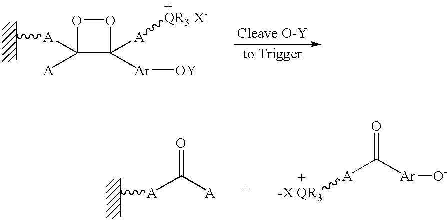
In the alternative, the linked onium group can be attached to the aryl group Ar or to the cleavable group Y. In a further alternative, the linkages to the solid phase and ternary or quaternary onium groups are reversed from the orientation shown.

Another cleavable linker group is an electron-rich C—C double bond which can be converted to an unstable 1,2-dioxetane moiety. At least one of the substituents on the double bond is attached to the double bond by means of an O, S, or N atom. Reaction of electron-rich double bonds with singlet oxygen produces an unstable 1,2-dioxetane ring group which spontaneously fragments at ambient temperatures to generate two carbonyl fragments. Unstable dioxetanes formed from electron-rich double bonds are described in numerous patents and publications exemplified by A. P. Schaap and S. D. Gagnon, J. Am. Chem. Soc., 104, 3504-6 (1982); W. Adam, Chem. Ber., 116, 839-46, (1983); U.S. Pat. No. 5,780,646.

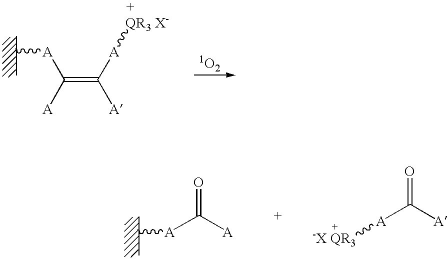

Another group of solid phase materials having a cleavable linker group have as the cleavable moiety a ketene dithioacetal as disclosed in PCT Publication WO 03/053934. Ketene dithioacetals undergo oxidative cleavage by enzymatic oxidation with a peroxidase enzyme and hydrogen peroxide. 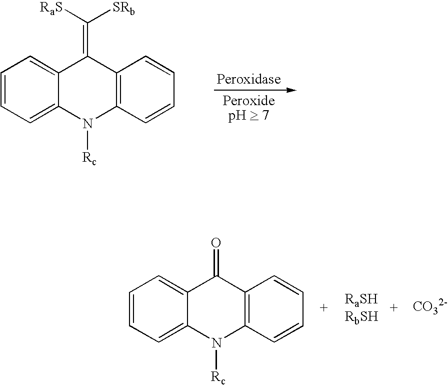
The cleavable moiety has the structure shown, including analogs having substitution on the acridan ring, wherein Rₐ R_{b} and R_{c} are each organic groups containing from 1 to about 50 non-hydrogen atoms selected from C, N, O, S, P, Si and halogen atoms and wherein Rₐ and R_{b} can be joined together to form a ring. Numerous other cleavable groups will be apparent to the skilled artisan.

The methods of solid phase nucleic acid capture can be put to numerous uses. As shown in the particular examples below, both single stranded and double stranded nucleic acid can be captured and released. DNA, RNA, and PNA can be captured and released.

A preferred use is in isolation of DNA from whole blood. DNA is extracted from leucocytes in a commonly used technique. Blood is typically treated to selectively lyse erythrocytes and after a precipitation or centrifugation step, the intact leucocytes are separately lysed to expose the nucleic acid content. Proteins are digested and the DNA obtained is isolated with a solid phase then used for determination of sequence polymorphism, sequence analysis, RFLP analysis, mutation detection or other types of diagnostic assay.

Another use is in isolating DNA from mixtures of DNA and RNA. Methods of the present invention involving strongly alkaline elution conditions, especially those using elevated temperatures, can degrade or destroy RNA present while leaving DNA intact. Methods involving strongly alkaline cleavage reactions will act similarly.

Additional uses include extraction of nucleic acid material from other samples—soil, plant, bacteria, and waste water and long term storage of nucleic acid materials for archival purposes.

An important advantage of the present methods is that they are compatible with many downstream molecular biology processes. Nucleic acid isolated by the present methods can in many cases be used directly in a further process. Amplification reactions such as PCR, Ligation of Multiple Oligomers (LMO) described in U.S. Pat. No. 5,998,175, and LCR can employ such nucleic acid eluents. Nucleic acid isolated by conventional techniques, especially from bacterial cell culture or from blood samples, employ a precipitation step. Low molecular weight alcohols are added in high volume percent to precipitate nucleic acid from aqueous solutions. The precipitated materials must then be separated, collected and redissolved in a suitable medium before use. These steps can be obviated by elution of nucleic acid from solid phase binding materials of the present invention using the present methods. It is a preferred practice to use the solution containing the released nucleic acid directly in a nucleic acid amplification reaction whereby the amount of the nucleic acid or a segment thereof is amplified using a polymerase or ligase-mediated reaction.

### EXAMPLES

Structure drawings when present in the examples below are intended to illustrate only the cleavable linker portion of the solid phase materials. The drawings do not represent a full definition of the solid phase material.

### Example 1

### Synthesis of Magnetic Silica Particles Functionalized with Polymethacrylate Linker and Containing Tributylphosphonium Groups and Cleavable Arylthioester Linkage

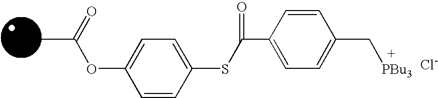

Magnetic carboxylic acid-functionalized silica particles (Chemicell, SiMAG-TCL, 1.0 meq/g, 1.5 g) were placed in 20 mL of thionyl chloride and refluxed for 4 hours. The excess thionyl chloride was removed under reduced pressure. The resin was resuspended in 25 mL of CHCl₃ and the suspension dispersed by ultrasound. The solvent was evaporated and ultrasonic wash treatment repeated. The particles were dried under vacuum for further use.

The acid chloride functionalized particles were suspended in 38 mL of CH₂Cl₂ along with 388 mg of diisopropylethylamine. 4′-Hydroxyphenyl 4-chloromethyl-thiobenzoate (524 mg) was added and the sealed reaction flask left on the shaker over night. The particles were transferred to a 50 mL plastic tube and washed repeatedly, with magnetic separation, with portions of CH₂Cl₂, CH₃OH, 1:1 CH₂Cl₂/CH₃OH, and then CH₂Cl₂. Wash solutions were monitored by TLC for removal of unreacted soluble starting materials. The solid was air dried before further use.

The resin (1.233 g) was suspended in 20 mL of CH₂Cl₂ under argon. Tributylphosphine (395 mg) was added and the slurry shaken for 7 days. The particles were transferred to a 50 mL plastic tube and washed 4 times with 40 mL of CH₂Cl₂ followed with 4 washes of 40 mL of MeOH and 4 times with 40 mL of CH₂Cl₂. The resin was then air dried yielding 1.17 g of a light brown solid.

### Example 2

### Synthesis of Silica Particles Functionalized with a Cleavable Linker Containing Tributylphosphonium Groups

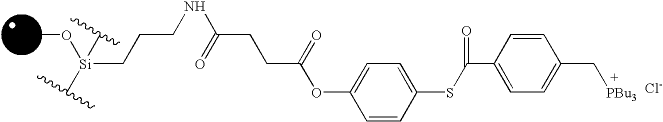

A solution of 3-aminopropyltriethoxysilane (13.2 mL) in 75 mL of heptane and 13 mL of ethanol was placed under Ar and stirred with 5.5 g of succinic anhydride. The reaction was refluxed for 4.5 h and then cooled to room temperature over night. The solvent was removed yielding the amide product as a clear oil.

A solution of EDC hydrochloride (4.0 g) and 2.86 g of the product above in 100 mL of CH₂Cl₂ was placed under Ar and stirred for 1 h before adding 4.16 g of 5.5 g of 4′-hydroxy-phenyl 4-chloromethylthiobenzoate. The reaction was stirred over night. The reaction mixture was chromatographed onto 150 g of silica, eluted with 1-2% EtOH/CH₂Cl₂ yielding 1.84 g of the coupled product as a white solid.

The product of the previous step (1.84 g) in 50 mL of dry toluene was added via cannula to a flask containing 3.83 g of oven-dried silica under a blanket of Ar. The reaction was refluxed over night. After cooling to room temperature, the silica was filtered off, washed with 500 mL of CH₂Cl₂, and vacuum dried for 4 h.

The derivatized silica having chlorobenzyl end groups (2.0 g) in 50 mL of CH₂Cl₂ was mixed with 8.0 g of tributylphosphine. The reaction mix was stirred under Ar for 2 d. The silica was filtered off, washed with CH₂Cl₂ and hexanes, and vacuum dried for several hours.

### Example 3

### Synthesis of a Magnetic Silica Particles Coated with a Cleavable Linker Containing Tributylphosphonium Groups

A nucleic acid binding material was prepared by passively adsorbing a cleavable nucleic acid binding group onto the surface of silica particles.

A 3 L flask was charged with 100.9 g of 4-chloromethyl-benzoic acid and 1.2 L of SOCl₂. the reaction was refluxed for 4 h, after which the thionyl chloride was removed under reduced pressure. Residual SOCl₂ was removed by addition of CH₂Cl₂ and evaporation under reduced pressure.

A 3 L flask containing 113.1 g of 4-chloromethylbenzoic acid chloride was charged with 98.17 g of 4-hydroxy-thiophenol and 1.5 L of CH₂Cl₂. Argon was purged in and 67.75 mL of pyridine added. After stirring over night, the reaction mixture diluted with 1 L of CH₂Cl₂ and extracted with 5 L of water. The water layer was back extracted with CH₂Cl₂. The combined CH₂Cl₂ solutions were dried over sodium sulfate and concentrated to a solid. The solid was washed with 500 mL of CH₂Cl₂₁ filtered and air dried. ¹H NMR (acetone-d₆): δ 4.809 (s, 2H), 6.946-6.968 (d, 2H), 7.323-7.346 (d, 2H), 7.643-7.664 (d, 2H), 8.004-8.025 (d, 2H).

Stearic acid (1.33 g) was refluxed in 10 mL of SOCl₂ for 2 h. The excess SOCl₂ was removed under reduced pressure producing stearoyl chloride as a brown liquid.

Stearoyl chloride was dissolved in 10 mL of CH₂Cl₂ and added to a solution of 1.0 g of 4′-hydroxyphenyl 4-chloro-methylthiobenzoate and 1.56 mL of diisopropylethylamine in 30 mL of CH₂Cl₂ and the mixture stirred over night. The solvent was removed and residue subject to column chromatography using 1:1 hexane/CH₂Cl₂ as eluent. The stearoyl ester (1.43 g) was isolated as a white solid.

A solution of the above product (1.43 g) and tributylphosphine (1.27 mL) in 30 mL of CH₂Cl₂ was stirred under an Ar atmosphere for 2 d. After removal of CH₂Cl₂ the residue was washed with 6×50 mL of ether, redissolved in CH₂Cl₂ and precipitated with ether producing 1.69 g of the phosphonium salt product. This material was found to be insoluble in water.

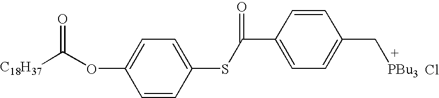

The phosphonium salt (0.6 g) was dissolved in 6 mL of CH₂Cl₂ and added to 6.0 g of silica gel with agitation. Evaporation of solvent produced the nucleic acid binding material.

### Example 4

### Synthesis of Magnetic Particle having a Polymeric Layer Containing Polyvinylbenzyl Tributylphosphonium Groups

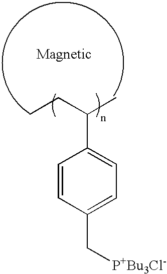

Magnetic Merrifield peptide resin (Chemicell, SiMag Chloromethyl, 100 mg) was added to 2 mL of CH₂Cl₂ in a glass vial. Tributylphosphine (80 μL) was added and the slurry was shaken at room temperature for 3 days. A magnet was placed under the vial and the supernatant was removed with a pipet. The solids were washed four times with 2 mL of CH₂Cl₂ (the washes were also removed by the magnet/pipet procedure). The resin was air dried (93 mg).

### Example 5

### Synthesis of Polymethacrylate Polymer Particles Containing Yributylphosphonium Groups and Cleavable Arylthioester Linkage

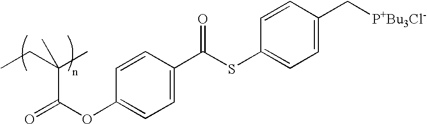

Poly(methacryloyl chloride) particles (1.0 meq/g, 1.5 g) were placed in 75 mL of CH₂Cl₂ containing 2.45 g of diisopropylethylamine. Triethylamine (1.2 g) was added. 4′-Hydroxyphenyl 4-chloromethylthiobenzoate (4.5 g) was added and the sealed reaction mixture was stirred overnight at room temperature. The slurry was filtered and the resin washed with 10 mL of CH₂Cl₂, 200 mL of acetone, 200 mL of MeOH, 2×100 mL of 1:1 THF/CH₂Cl₂, 250 mL of THF, 250 mL of CH₂Cl₂, 250 mL of hexane. The resin was air dried for further use.

The resin (1.525 g) was suspended in 25 mL of CH₂Cl₂ under argon. Tributylphosphine (1.7 g) was added and the slurry stirred for 4 days. The resin was filtered and washed 4 times with 225 mL of CH₂Cl₂ followed by 175 mL of hexane. The resin was then air dried yielding 1.68 g of solid.

### Example 6

### Synthesis of a Polystyrene Polymer Containing Tributylphosphonium Groups

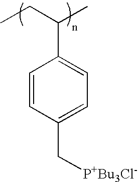

Merrifield peptide resin (Sigma, 1.1 meq/g, 20.0 g) which is a crosslinked chloromethylated polystyrene was stirred in 200 mL of CH₂Cl₂/DMF (50/50) under an argon pad. An excess of tributylphosphine (48.1 g, 10 equivalents) was added and the slurry was stirred at room temperature for 7 days. The slurry was filtered and the resulting solids were washed twice with 200 mL of CH₂Cl₂. The resin was dried under vacuum (21.5 g). Elemental Analysis: Found P 2.52%, Cl 3.08%; Expected P 2.79%, Cl 3.19%: P/Cl ratio is 0.94.

### Example 7

### Synthesis of a Polystyrene Polymer Containing Tributylammonium Groups

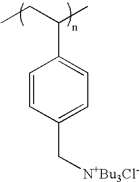

Merrifield peptide resin (Aldrich, 1.43 meq/g, 25.1 g) was stirred in 150 mL of CH₂Cl₂ under an argon pad. An excess of tributyl amine (25.6 g, 4 equivalents) was added and the slurry was stirred at room temperature for 8 days. The slurry was filtered and the resulting solids were washed twice with 250 mL of CH₂Cl₂. The resin was dried under vacuum (28.9 g). Elemental Analysis: Found N 1.18%, Cl 3.40%; Expected N 1.58%, Cl 4.01%: N/Cl ratio is 0.88.

### Example 8

### Synthesis of Silica Particles Functionalized with Tributylphosphonium Groups

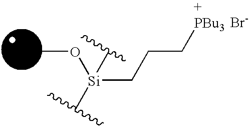

Silica gel dried for 1 h at 110° C. under Ar (4.82 g) was added to 50 mL of CH₂Cl₂ along with 2.79 g of Et₃N. The mixture was stirred for 20 min after which 2.56 g of 3-bromopropyltrichlorosilane was added, causing an exotherm. The mixture was stirred for 24 h, filtered and the solid washed sequentially with 3×40 mL of CH₂Cl₂, 4×40 mL of MeOH and 2×40 mL of CH₂Cl₂. The solid was air-dried over night and weighed 6.13 g.

The functionalized silica prepared above (5.8 g) in 50 mL of CH₂Cl₂ was stirred with 5.33 mL of tributylphosphine for 10 days. The mixture filtered and the solid washed with 7×50 mL of acetone. Air drying the solid produced 5.88 g of the product.

### Example 9

### Controlled Cleavage of Linker in NAB Material of Example 3

The coated silica material of example 3 (70 mg) was suspended in 1.0 mL of D₂O and mixed by vortexing for 3 min. Analysis of the water solution by ¹H NMR showed no release of material into solution.

Treatment of the silica suspension with 40 AL of 40% NaOD and vortexing for 3 min and NMR analysis of the supernatant showed cleavage of the linker and release from the silica into solution.

### Example 10

### Capture of DNA from Whole Human Blood

A 10 mg portion of the particles of each of examples 1-8 was mixed with 70 μL of whole human blood in a tube. the tube was vortex mixed for 15 s, held for 5 min at room temperature and again vortex mixed for 15 s. The mixture was diluted with 300 μL of 10 mM tris buffer, pH 8.0 and the liquid removed from the particles, with the aid of a magnet when magnetically responsive particles were employed.

### Example 11

### Isolation of DNA from Whole Human Blood

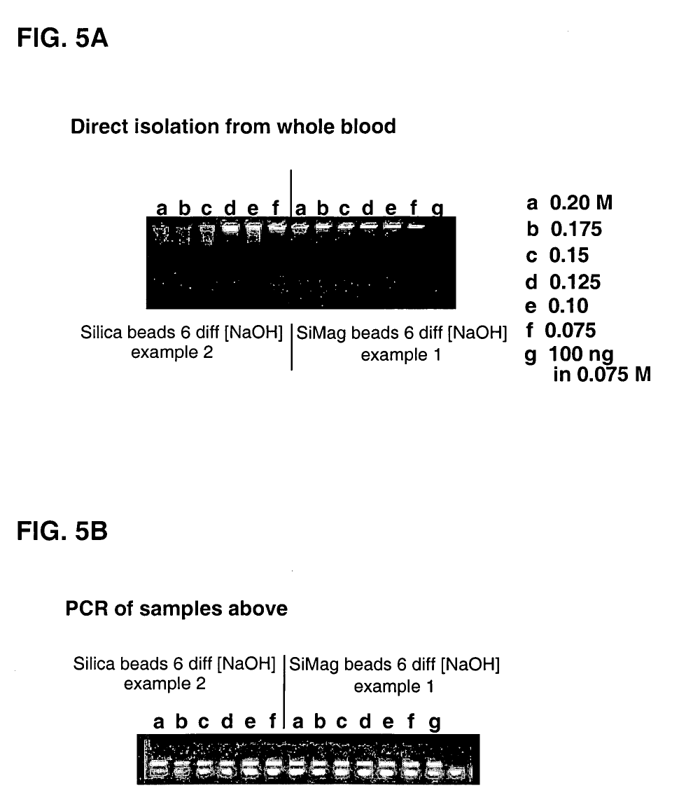

Nucleic acid captured on the solid phase binding material according to the procedure of the preceding example was washed three times with 500 μL of 10 mM tris buffer, pH 8.0, discarding the supernatant each time. Nucleic acids was removed from the particles by eluting with 100 μL of 0.1 M NaOH at 37° C. for 5 min. Other concentrations of NaOH were also effective as shown in FIGS. 5A and 5B.

### Example 12

### PCR Amplification of Genomic DNA

The eluted DNA of the previous example (1 μL) in 0.1 M NaOH was subject to PCR amplification with a pair of 24 base primers which produced a 200 bp amplicon. PCR reaction mixtures contained the components listed in the table below.

| Component | volume (μL) |
|---|---|
| 10X PCR buffer | 2 |
| Primer 1 (100 ng/μL) | 2 |
| Primer 2 (100 ng/μL) | 2 |
| 2.5 mM dNTPs | 2 |
| 50 mM MgCl₂ | 1.25 |
| Taq DNA polymerase (5 U/μL) | 0.25 |
| Template | 1 |
| deionized water | 9.5 |
| Total | 20 |

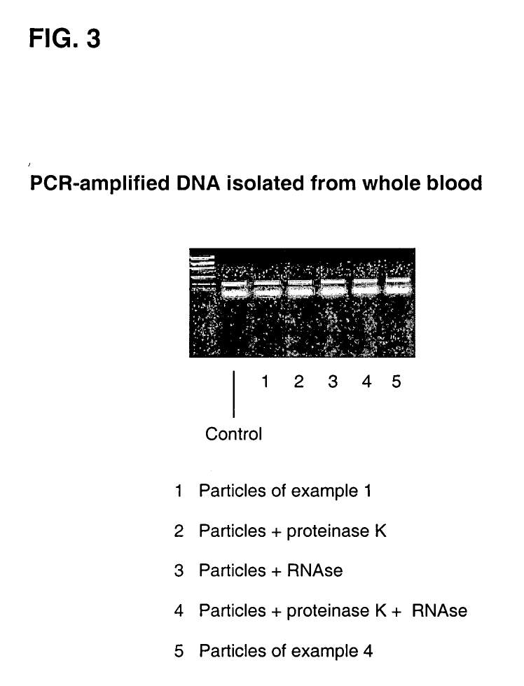

Negative controls replaced template in the reaction mix with 1 μL of water. A further reaction used 1 μL of template diluted 1:10 in water. Reaction mixtures were subject to 30 cycles of 94° C., 30 s; 60° C., 30 s; 72° C., 30 s. Reaction products were run on 1.5% agarose gel. FIG. 3 demonstrates that the DNA eluted from the beads is intact.

1. A method of isolating nucleic acids from a sample of cellular material comprising:
a) providing a solid phase comprising: a matrix to which is attached a nucleic acid binding portion;
b) combining the solid phase with a sample of cellular material containing nucleic acids in the absence of any added lysis solution for a time sufficient to bind the nucleic acids to the solid phase;
c) separating the sample from the solid phase; and
d) releasing the bound nucleic acids from the solid phase.

2. The method of claim 1 wherein the cellular material is selected from the group consisting of intact cells of animal, plant or bacterial origin and tissue containing intact cells of animal, plant or bacterial origin.

3. The method of claim 1 wherein the sample is selected from the group consisting of bacterial cultures, bodily fluids, whole blood and blood components, tissue extracts, plant materials, and environmental samples containing cellular materials.

4. The method of claim 1 wherein the sample is whole blood.

5. The method of claim 1 wherein the nucleic acid is selected from the group consisting of DNA and RNA.

6. The method of claim 5 wherein the nucleic acid is genomic DNA of an organism.

7. The method of claim 5 wherein the nucleic acid is genomic DNA of a human obtained from whole blood.

8. The method of claim 1 wherein the matrix is selected from silica, glass, insoluble synthetic polymers, and insoluble polysaccharides.

9. The method of claim 1 wherein the matrix of the solid phase is silica.

10. The method of claim 1 wherein the solid phase further comprises a magnetically responsive portion.

11. The method of claim 1 wherein the nucleic acid binding portion is selected from the group consisting of ternary sulfonium groups, quaternary ammonium groups and quaternary phosphonium groups.

12. The method of claim 1 wherein the nucleic acid binding portion is attached to the matrix through a linkage which can be selectively cleaved.

13. The method of claim 1 wherein the bound nucleic acids are released from the solid phase is a strongly alkaline solution.
